(19) United States
(12) Patent Application Publication
(10) Pub. No.: US 2026/0346051 A1
(43) Pub. Date: Nov. 5, 2026

(54) RADIATION TREATMENT PLAN OPTIMIZATION QUEUEING METHOD AND APPARATUS

(71) Applicant: Varian Medical Systems, Inc., Palo Alto, CA (US)

(72) Inventors: Michael Brewer, Dyer, IN (US); Jonathan Rogers, Fort Mill, SC (US)

(21) Appl. No.: 19/663,389

(22) Filed: Apr. 30, 2026

Related U.S. Application Data
(60) Provisional application No. 63/800,145, filed on May 5, 2025.

(51) Int. Cl. A61N 5/10

(52) U.S. Cl. CPC A61N 5/1048; A61N 5/1031; A61N 2005/1074

(57) ABSTRACT

A control circuit, upon populating an automatic radiation treatment planner with optimization parameters for the radiation treatment plan, presents a user, via a user interface, with an opportunity to place optimization of the radiation treatment plan as a function of the optimization parameters into an execution queue.

### CROSS-REFERENCE TO RELATED APPLICATION

This application claims benefit of U.S. Provisional Application No. 63/800,145, filed May 5, 2025, which is hereby incorporated herein by reference in its entirety.

### TECHNICAL FIELD

These teachings relate generally to treating a patient’s planning target volume with energy pursuant to an energy-based treatment plan and more particularly to optimizing an energy-based treatment plan.

### BACKGROUND

The use of energy to treat medical conditions comprises a known area of prior art endeavor. For example, radiation therapy comprises an important component of many treatment plans for reducing or eliminating unwanted tumors. Unfortunately, applied energy does not inherently discriminate between unwanted material and adjacent tissues, organs, or the like that are desired or even critical to continued survival of the patient. As a result, energy such as radiation is ordinarily applied in a carefully administered manner to at least attempt to restrict the energy to a given target volume. A so-called radiation treatment plan often serves in the foregoing regards.

A radiation treatment plan typically comprises specified values for each of a variety of treatment-platform parameters during each of a plurality of sequential fields. Treatment plans for radiation treatment sessions are often automatically generated through a so-called optimization process. As used herein, “optimization” will be understood to refer to improving a candidate treatment plan without necessarily ensuring that the optimized result is, in fact, the singular best solution. Such optimization often includes automatically adjusting one or more physical treatment parameters (often while observing one or more corresponding limits in these regards) and mathematically calculating a likely corresponding treatment result (such as a level of dosing) to identify a given set of treatment parameters that represent a good compromise between the desired therapeutic result and avoidance of undesired collateral effects.

Generating a radiation treatment plan often involves a sequence of steps including the foregoing optimization. Each step—such as plan creation, beam setup, optimization, and dose calculation—may require some degree of user intervention. Some available systems streamline this process by automating sequences of these steps.

### BRIEF DESCRIPTION OF DRAWINGS

Various needs are at least partially met through provision of the radiation treatment plan optimization queueing method and apparatus described in the following detailed description, particularly when studied in conjunction with the drawings, wherein:

FIG. 1 comprises a block diagram as configured in accordance with various embodiments of these teachings;

FIG. 2 comprises a flow diagram as configured in accordance with various embodiments of these teachings;

FIG. 3 comprises a schematic view of a display screen as configured in accordance with various embodiments of these teachings;

FIG. 4 comprises a screen shot as configured in accordance with various embodiments of these teachings; and

FIG. 5 comprises a screen shot as configured in accordance with various embodiments of these teachings.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein. The word “or” when used herein shall be interpreted as having a disjunctive construction rather than a conjunctive construction unless otherwise specifically indicated.

### DETAILED DESCRIPTION

Generally speaking, these teachings can provide for optimizing a radiation treatment plan for a particular patient using a particular radiation treatment machine at a corresponding treatment facility. By one approach, a control circuit, upon populating an automatic radiation treatment planner with optimization parameters for the radiation treatment plan, presents a user, via a user interface, with an opportunity to place optimization of the radiation treatment plan as a function of the optimization parameters into an execution queue. If desired, the user may also be provided with an alternative opportunity to begin optimization of the radiation treatment plan as a function of the optimization parameters without use of the execution queue. (As used herein, it will be understood that the word “execution” refers to executing the steps of generating a radiation treatment plan, and not to administering a radiation treatment plan to a patient.)

By one approach, the execution queue can be configured to automatically execute optimization of each of a plurality of radiation treatment plans that are assigned to the execution queue. That execution of a plurality of radiation treatment plans may proceed in seriatim fashion or, if desired, may be conducted wholly or partially in parallel with one another as desired.

By one approach, the execution queue can be configured to automatically execute optimization of at least one of the plurality of radiation treatment plans that are assigned to the execution queue as a background processing task. By another approach, the execution queue can be configured to automatically execute optimization of at least one of the plurality of radiation treatment plans that are assigned to the execution queue during non-working hours for the treatment facility. If desired, the user may be provided with an opportunity to select between the foregoing choices and/or other choices that may be available.

By one approach, the execution queue can be configured to automatically execute optimization of each of a plurality of radiation treatment plans that are assigned to the execution queue in a prioritized sequence of execution. In such a case, and by one approach, the opportunity to place optimization of the radiation treatment plan as a function of the optimization parameters into an execution queue can include an opportunity to selectively place optimization of the radiation treatment plan into a particular level of the execution queue (where, for example, that particular level comprises a first-in-line-to-be-executed level of the execution queue).

These teachings are highly flexible in practice and will, for example, accommodate the execution queue corresponding only to radiation treatment plans that are to be executed for only a single user. If desired, however, these teachings will accommodate the execution queue queueing and executing radiation treatment plans for at least two users for the treatment facility.

By one approach, these teachings can comprise a non-transitory computer-readable medium having a computer program that itself comprises instructions that, when the computer program is executed by a computer, causes the computer to carry out any of the steps, activities, and/or functions described herein including but not limited to, upon populating an automatic radiation treatment planner with optimization parameters for the radiation treatment plan, presenting a user, via a user interface, with an opportunity to place optimization of the radiation treatment plan as a function of the optimization parameters into an execution queue.

So configured, these teachings will permit leveraging the automation capabilities of a radiation treatment plan optimization system to effectively extend those capabilities beyond use with a single plan. In particular, instead of generating just one plan, one or more users can populate a queue with multiple sequences that, when executed, generate a batch of plans automatically.

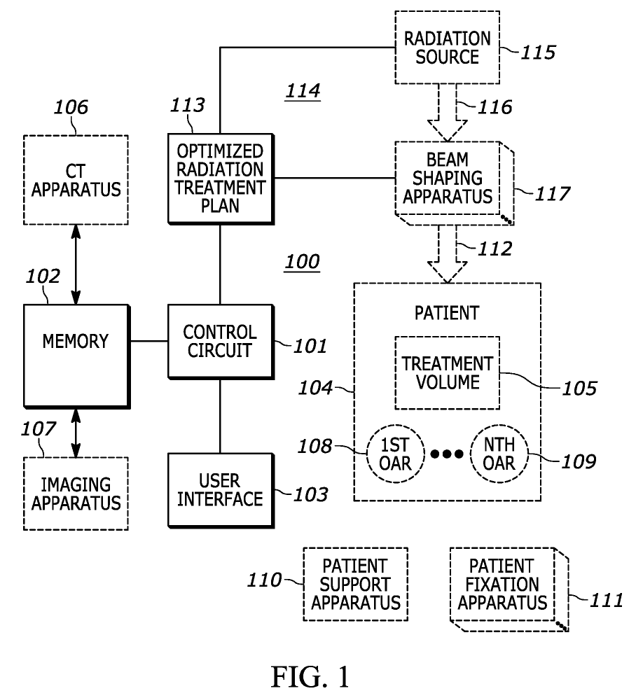

These and other benefits may become clearer upon making a thorough review and study of the following detailed description. Referring now to the drawings, and in particular to FIG. 1, a treatment facility 100 that is compatible with many of these teachings will first be presented.

In this particular example, the treatment facility 100 includes a control circuit 101. Being a “circuit,” the control circuit 101 therefore comprises structure that includes at least one (and typically many) electrically-conductive paths (such as paths comprised of a conductive metal such as copper or silver) that convey electricity in an ordered manner, which path(s) will also typically include corresponding electrical components (both passive (such as resistors and capacitors) and active (such as any of a variety of semiconductor-based devices) as appropriate) to permit the circuit to effect the control aspect of these teachings.

Such a control circuit 101 can comprise a fixed-purpose hard-wired hardware platform (including but not limited to an application-specific integrated circuit (ASIC) (which is an integrated circuit that is customized by design for a particular use, rather than intended for general-purpose use), a field-programmable gate array (FPGA), and the like) or can comprise a partially or wholly-programmable hardware platform (including but not limited to microcontrollers, microprocessors, and the like). These architectural options for such structures are well known and understood in the art and require no further description here. This control circuit 101 is configured (for example, by using corresponding programming as will be well understood by those skilled in the art) to carry out one or more of the steps, actions, and/or functions described herein.

It will be appreciated that the control circuit 101 may comprise a single integrated platform or may comprise a plurality of such circuits that work in cooperation with one another.

The control circuit 101 operably couples to a memory 102. This memory 102 may be integral to the control circuit 101 or can be physically discrete (in whole or in part) from the control circuit 101 as desired. This memory 102 can also be local with respect to the control circuit 101 (where, for example, both share a common circuit board, chassis, power supply, and/or housing) or can be partially or wholly remote with respect to the control circuit 101 (where, for example, the memory 102 is physically located in another facility, metropolitan area, or even country as compared to the control circuit 101). As with the control circuit 101, the memory 102 may comprise a singular structure or may comprise a plurality of memory platforms that collectively comprise the “memory” of this apparatus 100.

In addition to information such as optimization information for a particular patient and information regarding a particular radiation treatment platform as described herein, this memory 102 can serve, for example, to non-transitorily store the computer instructions that, when executed by the control circuit 101, cause the control circuit 101 to behave as described herein. (As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM) as well as volatile memory (such as a dynamic random access memory (DRAM))).

In this illustrative example, the control circuit 101 also operably couples to a user interface 103. This user interface 103 can comprise any of a variety of user-input mechanisms (such as, but not limited to, keyboards and keypads, cursor-control devices, touch-sensitive displays, speech-recognition interfaces, gesture-recognition interfaces, and so forth) and/or user-output mechanisms (such as, but not limited to, visual displays, audio transducers, printers, and so forth) to facilitate receiving information and/or instructions from a user and/or providing information to a user.

If desired the control circuit 101 can also operably couple to a network interface (not shown). So configured the control circuit 101 can communicate with other elements (both within the treatment facility 100 and external thereto) via the network interface. Network interfaces, including both wireless and non-wireless platforms, are well understood in the art and require no particular elaboration here.

By one approach, a computed tomography apparatus 106 and/or other imaging apparatus 107 as are known in the art can source some or all of any desired patient-related imaging information.

In this illustrative example the control circuit 101 is configured to ultimately output an optimized energy-based treatment plan (such as, for example, an optimized radiation treatment plan 113). This energy-based treatment plan typically comprises specified values for each of a variety of treatment-platform parameters during each of a plurality of sequential exposure fields. In this case the energy-based treatment plan is generated through an optimization process.

By one approach the control circuit 101 can operably couple to an energy-based treatment platform 114 that is configured to deliver therapeutic energy 112 to a corresponding patient 104 having at least one treatment volume 105 and also one or more organs-at-risk (represented in FIG. 1 by a first through an Nth organ-at-risk 108 and 109) in accordance with the optimized energy-based treatment plan 113. These teachings are generally applicable for use with any of a wide variety of energy-based treatment platforms/apparatuses. In a typical application setting the energy-based treatment platform 114 will include an energy source such as a radiation source 115 of ionizing radiation 116.

By one approach, this radiation source 115 can be selectively moved via a gantry along an arcuate pathway (where the pathway encompasses, at least to some extent, the patient themselves during administration of the treatment). The arcuate pathway may comprise a complete or nearly complete circle as desired. By one approach the control circuit 101 controls the movement of the radiation source 115 along that arcuate pathway, and may accordingly control when the radiation source 115 starts moving, stops moving, accelerates, de-accelerates, and/or a velocity at which the radiation source 115 travels along the arcuate pathway.

As one illustrative example, the radiation source 115 can comprise, for example, a radio-frequency (RF) linear particle accelerator-based (linac-based) x-ray source. A linac is a type of particle accelerator that greatly increases the kinetic energy of charged subatomic particles or ions by subjecting the charged particles to a series of oscillating electric potentials along a linear beamline, which can be used to generate ionizing radiation (e.g., X-rays) 116 and high energy electrons.

A typical energy-based treatment platform 114 may also include one or more support apparatuses 110 (such as a couch) to support the patient 104 during the treatment session, one or more patient fixation apparatuses 111, a gantry or other movable mechanism to permit selective movement of the radiation source 115, and one or more energy-shaping apparatuses (for example, beam-shaping apparatuses 117 such as jaws, multi-leaf collimators, and so forth) to provide selective energy shaping and/or energy modulation as desired.

In a typical application setting, it is presumed herein that the patient support apparatus 110 is selectively controllable to move in any direction (i.e., any X, Y, or Z direction) during an energy-based treatment session by the control circuit 101. As the foregoing elements and systems are well understood in the art, further elaboration in these regards is not provided here except where otherwise relevant to the description.

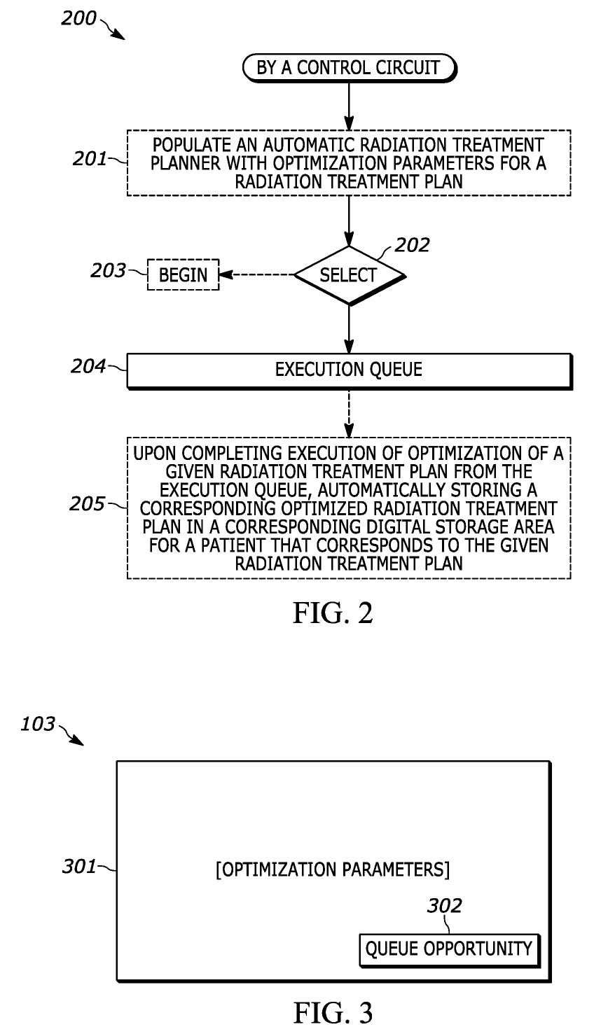

Referring now to FIGS. 2 and 3, a process 200 that can be carried out, for example, in conjunction with the above-described application setting (and more particularly via the aforementioned control circuit 101) will be described. Generally speaking, this process 200 serves to facilitate optimizing a radiation treatment plan for a particular patient 104 using a particular radiation treatment machine 114 at a corresponding treatment facility 100.

At optional block 201, this process 200 can provide for populating an automatic radiation treatment planner with optimization parameters 301 for a corresponding radiation treatment plan. Parameters in these regards can include, but are not limited to, such things as radiation treatment machine identifiers, patient support identifiers, dosage prescription information, target details, and selections of particular dose models or optimization models, amongst many other possibilities. In this illustrative embodiment, these optimization parameters 301 appear on a display screen that comprises a part of the above-mentioned user interface 103. One or more screens may be utilized to present all optimization parameters 301. Depending upon the application setting, one or more of these optimization parameters 301 may comprise default values, automatically-selected values, and/or values and entries selected or entered by the user.

In this illustrative example, the control circuit 101 presents an opportunity, such as a displayed soft button that can be selected by the user, to select a queue opportunity 302. By one optional approach, and as shown in FIG. 2 at blocks 202 and 203, the user can also be presented with an opportunity to select to currently begin to optimize the radiation treatment plan as a function of those populated optimization parameters 301 without first being queued. In the latter case, optimization can begin in the foregoing (as versus the background as described below).

In lieu of the foregoing, or in combination there with, selecting the queue opportunity 302 in this illustrative example serves to place optimization of the current radiation treatment plan as a function of the aforementioned optimization parameters 301 into an execution queue 204.

By one approach, the execution queue 204 is configured to automatically execute optimization of each of a possible plurality of radiation treatment plans that have been assigned to that execution queue 204.

By one approach, that execution may comprise executing the optimization as a background processing task. Background processing refers to computational tasks that are executed by a system in a manner that does not interfere with or require direct interaction from the user during the user’s other primary computer-usage activities. In the context of a radiation treatment planning system, background processing allows the optimization of the treatment plan to occur efficiently without disrupting the user’s ability to review, modify, or interact with other aspects of the system.

By another approach, in lieu of the foregoing or in combination therewith, execution of the optimization task can be scheduled to occur during non-working hours for the treatment facility 100. If desired, the aforementioned queue opportunity 302 can include an opportunity for the user to select background processing as versus processing during non-working hours or vice versa.

By one approach, the execution queue 204 may be configured to only permit execution of the queued activities using a first-in/first-out prioritization scheme. By one approach, the latter prioritization scheme may be modified to permit the user to selectively place optimization of a radiation treatment plan into a particular level of the execution queue, such as a first-in-line-to-be-executed level of the execution queue 204.

By one approach, the execution queue 204 corresponds to only a single user. So configured, only radiation treatment plans as input by that one user are queued in that execution queue 204. By another approach, the execution queue 204 can contain queued radiation treatment plans for more than a single user. For example, at least two users for the treatment facility 100 may have one or more radiation treatment plans placed in the execution queue 204 to await execution.

By one approach, and as illustrated at optional block 205, this process 200 can additionally provide for, upon completing execution of optimization of a given radiation treatment plan from the execution queue 204, then automatically storing a corresponding resultant optimized radiation treatment plan in a corresponding digital storage area for a patient that corresponds to the given radiation treatment plan.

These teachings can support operating without user intervention once started. The foregoing process 200 can generate multiple candidate plans in the background (or during after hours), thereby allowing the user to focus on other tasks such as documentation, quality assurance, chart checks, or more complex planning. The resulting optimized plans may be ready for clinical review or, at the very least, serve as a solid starting point for subsequent planning activities, thereby reducing the time and attention needed to generate final treatment plans.

Following optimization and final acceptance, a resultant optimized radiation treatment plan 113 can then be administered to the particular patient 104 using the particular radiation treatment machine 114 at the particular facility 100.

Further details that comport with these teachings will now be presented. It will be understood that the specific details of these examples are intended to serve an illustrative purpose and are not intended to suggest any particular limitations with respect to these teachings.

By one approach, these teachings will support a workflow providing for a single sequence per plan. In this case, a planner assigns one sequence per planning task and adds each to the execution queue 204. The execution queue 104 can then be run in the background or overnight, producing corresponding candidate plans for subsequent review by the user and/or others.

By another approach, these teachings will support a workflow providing for multiple sequences per plan. In this case, the user can preconfigure multiple sequences for a single patient, to thereby accommodate, for example, varying arc counts or dose levels. For example, head and neck cases can require empirical testing between two-arc and three-arc plans. By employing the present teachings, a user can queue both options without further input, allowing for side-by-side comparison once both approaches have been executed. Variations may also include dose escalation, alternate models, and/or selective exclusion/inclusion of organs-at-risk.

By another approach, these teachings will support a workflow providing for centralized assignment and distributed review. In this case, as structure sets are completed, a user (who may be an assigned “super-user”) can assign one or more sequences to the execution queue 204. The execution queue 204 executes the queued input in the background or overnight. Once the batch run is complete, candidate plans can be distributed to each of a plurality of users for review and refinement. Such an approach can significantly reduce manual effort and can enable user to begin their planning with a head start rather than from scratch.

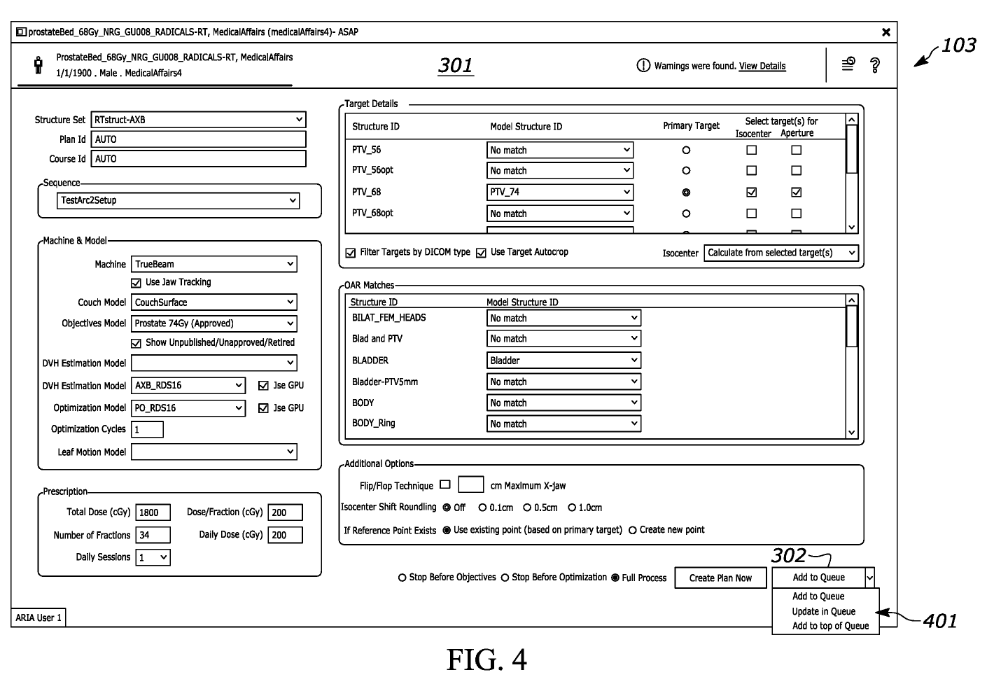

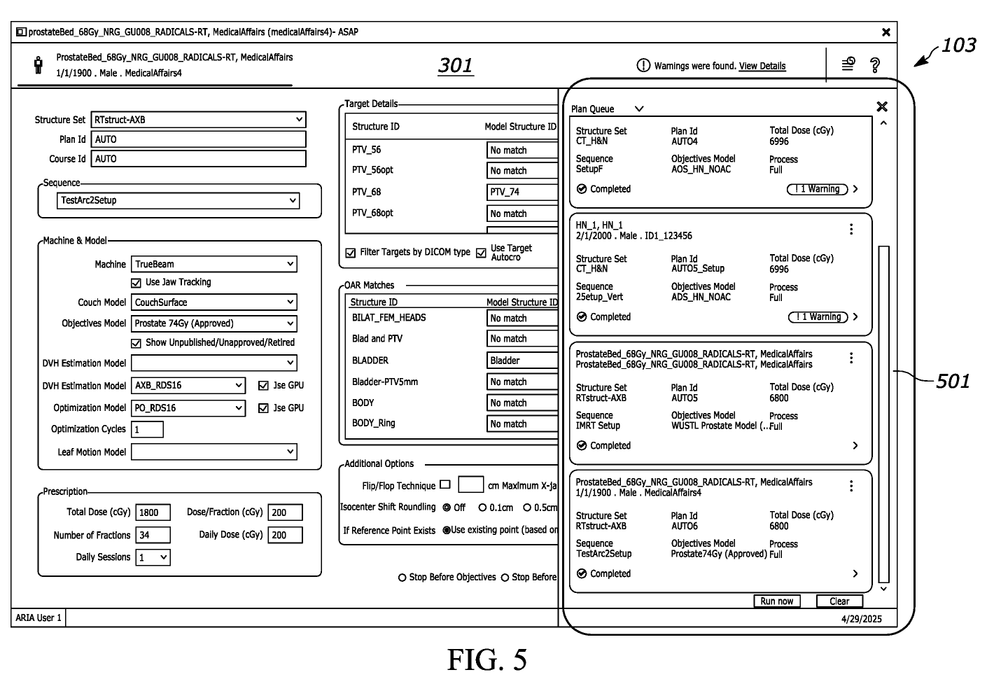

FIG. 4 presents an illustrative example in the foregoing regards. In this example, when the user clicks on the queue opportunity 302, a submenu 401 opens that provides various queuing options. In this example, those options include adding the current project to the execution queue 204 (for example, to the bottom of the existing list of queued plans), updating the queue (as described below in more detail with respect to FIG. 5) for matching patients, courses, and plan identifiers, and adding the current project to the top of the execution queue 204 and hence placing the plan into the execution queue 204 with a potentially higher priority than other already-queued plans. These teachings will of course accommodate other queuing options.

The update the queue option noted above can lead, by one approach, to the execution queue status window 501 shown in FIG. 5.By one approach, this option can permit a user to view progress messages that may be available. If desired, this option can provide for selecting an item in the execution queue 204 and loading that item back into the main user interface for review and/or editing. By one approach, the foregoing can include the option to "Update in Queue," which refers to updating the originating item (as versus adding a new one). By one approach, all parameters can be made eligible for update. For example, the user may load the item back into the user interface, change such things as the planning model, the treatment machine, and/or the prescription dose, and then update that revised item in the execution queue 204. By one approach, warnings and failures can be suppressed, and the user can see such content in this execution queue status window 501, while the queue will still run the remaining objects.

Further aspects of these teachings are provided by the subject matter of the following clauses (where it will be understood that any of these clauses can be combined with any one of more of the other clauses as desired).

Clause 1. A method for optimizing a radiation treatment plan for a particular patient using a particular radiation treatment machine at a corresponding treatment facility, the method comprising: by a control circuit: upon populating an automatic radiation treatment planner with optimization parameters for the radiation treatment plan, presenting a user, via a user interface, with an opportunity to: begin optimization of the radiation treatment plan as a function of the optimization parameters; or place optimization of the radiation treatment plan as a function of the optimization parameters into an execution queue.

Clause 2. The method of clause 1 wherein the execution queue is configured to automatically execute optimization of each of a plurality of radiation treatment plans that are assigned to the execution queue.

Clause 3. The method of clause 2 wherein the execution queue is configured to automatically execute optimization of at least one of the plurality of radiation treatment plans that are assigned to the execution queue as a background processing task.

Clause 4. The method of clause 2 wherein the execution queue is configured to automatically execute optimization of at least one of the plurality of radiation treatment plans that are assigned to the execution queue during non-working hours for the treatment facility.

Clause 5. The method of clause 2 wherein the execution queue is configured to automatically execute optimization of each of a plurality of radiation treatment plans that are assigned to the execution queue in a prioritized sequence of execution.

Clause 6. The method of clause 5 wherein the opportunity to place optimization of the radiation treatment plan as a function of the optimization parameters into an execution queue includes an opportunity to selectively place optimization of the radiation treatment plan into a particular level of the execution queue.

Clause 7. The method of clause 6 wherein the particular level comprises a first-in-line-to-be-executed level of the execution queue.

Clause 8. The method of clause 1 wherein the execution queue corresponds to radiation treatment plans to be executed for one of: a single user; at least two users for the treatment facility.

Clause 9. The method of clause 1 further comprising: upon completing execution of optimization of a given radiation treatment plan from the execution queue, automatically storing a corresponding optimized radiation treatment plan in a corresponding digital storage area for a patient that corresponds to the given radiation treatment plan.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

1. A method for optimizing a radiation treatment plan for a particular patient using a particular radiation treatment machine at a corresponding treatment facility, the method comprising:
by a control circuit:
upon populating an automatic radiation treatment planner with optimization parameters for the radiation treatment plan, presenting a user, via a user interface, with an opportunity to:
begin optimization of the radiation treatment plan as a function of the optimization parameters; or
place optimization of the radiation treatment plan as a function of the optimization parameters into an execution queue.

2. The method of claim 1 wherein the execution queue is configured to automatically execute optimization of each of a plurality of radiation treatment plans that are assigned to the execution queue.

3. The method of claim 2 wherein the execution queue is configured to automatically execute optimization of at least one of the plurality of radiation treatment plans that are assigned to the execution queue as a background processing task.

4. The method of claim 2 wherein the execution queue is configured to automatically execute optimization of at least one of the plurality of radiation treatment plans that are assigned to the execution queue during non-working hours for the treatment facility.

5. The method of claim 2 wherein the execution queue is configured to automatically execute optimization of each of a plurality of radiation treatment plans that are assigned to the execution queue in a prioritized sequence of execution.

6. The method of claim 5 wherein the opportunity to place optimization of the radiation treatment plan as a function of the optimization parameters into an execution queue includes an opportunity to selectively place optimization of the radiation treatment plan into a particular level of the execution queue.

7. The method of claim 6 wherein the particular level comprises a first-in-line-to-be-executed level of the execution queue.

8. The method of claim 1 wherein the execution queue corresponds to radiation treatment plans to be executed for one of:
a single user;
at least two users for the treatment facility.

9. The method of claim 1 further comprising:
upon completing execution of optimization of a given radiation treatment plan from the execution queue, automatically storing a corresponding optimized radiation treatment plan in a corresponding digital storage area for a patient that corresponds to the given radiation treatment plan.

10. An apparatus for optimizing a radiation treatment plan for a particular patient using a particular radiation treatment machine at a corresponding treatment facility, the apparatus comprising:
a control circuit configured to:
upon populating an automatic radiation treatment planner with optimization parameters for the radiation treatment plan, present a user, via a user interface, with an opportunity to:
begin optimization of the radiation treatment plan as a function of the optimization parameters; or
place optimization of the radiation treatment plan as a function of the optimization parameters into an execution queue.

11. The apparatus of claim 10 wherein the execution queue is configured to automatically execute optimization of each of a plurality of radiation treatment plans that are assigned to the execution queue.

12. The apparatus of claim 11 wherein the execution queue is configured to automatically execute optimization of at least one of the plurality of radiation treatment plans that are assigned to the execution queue as a background processing task.

13. The apparatus of claim 11 wherein the execution queue is configured to automatically execute optimization of at least one of the plurality of radiation treatment plans that are assigned to the execution queue during non-working hours for the treatment facility.

14. The apparatus of claim 11 wherein the execution queue is configured to automatically execute optimization of each of a plurality of radiation treatment plans that are assigned to the execution queue in a prioritized sequence of execution.

15. The apparatus of claim 14 wherein the opportunity to place optimization of the radiation treatment plan as a function of the optimization parameters into an execution queue includes an opportunity to selectively place optimization of the radiation treatment plan into a particular level of the execution queue.

16. The apparatus of claim 15 wherein the particular level comprises a first-in-line-to-be-executed level of the execution queue.

17. The apparatus of claim 10 wherein the execution queue corresponds to radiation treatment plans to be executed for one of:
a single user;
at least two users for the treatment facility.

18. The apparatus of claim 10 wherein the control circuit is further configured to:
upon completing execution of optimization of a given radiation treatment plan from the execution queue, automatically store a corresponding optimized radiation treatment plan in a corresponding digital storage area for a patient that corresponds to the given radiation treatment plan.

19. A non-transitory computer-readable medium comprising instructions stored thereon for optimizing a radiation treatment plan for a particular patient using a particular radiation treatment machine at a corresponding treatment facility, which instructions, when executed on a processor, perform the steps of:
upon populating an automatic radiation treatment planner with optimization parameters for the radiation treatment plan, presenting a user, via a user interface, with an opportunity to:
begin optimization of the radiation treatment plan as a function of the optimization parameters; or
place optimization of the radiation treatment plan as a function of the optimization parameters into an execution queue.

20. The non-transitory computer-readable medium of claim 19 wherein the execution queue is configured to automatically execute optimization of each of a plurality of radiation treatment plans that are assigned to the execution queue.
